(19) United States
(12) Patent Application Publication
(10) Pub. No.: US 2009/0162293 A1
(43) Pub. Date: Jun. 25, 2009

(54) METHODS AND COMPOSITIONS FOR ULTRASOUND IMAGING OF APOPTOSIS

(75) Inventors: Jeffrey D. Gabe, San Bruno, CA (US); Thomas B. Ottoboni, Belmont, CA (US)

Correspondence Address: DECHERT LLP, P.O. BOX 390460, MOUNTAIN VIEW, CA, 94039-0460, US

(73) Assignee: Point Biomedical Corporation, San Carlos, CA (US)

(21) Appl. No.: 12/196,107

(22) Filed: Aug. 21, 2008

Related U.S. Application Data
(63) Continuation of application No. 10/937,035, filed on Sep. 8, 2004, now abandoned.
(60) Provisional application No. 60/501,613, filed on Sep. 9, 2003.

(51) Int. Cl. A61B 8/00

(52) U.S. Cl. 424/9.52; 424/9.5

(57) ABSTRACT

In some aspects, there are provided compositions and kits including annexin coupled to ultrasound contrast particles as well as methods utilizing these particles for diagnosis and treatment of pathological conditions characterized by apoptosis.

### 1. CROSS-REFERENCE TO RELATED CO-PENDING APPLICATIONS

This application claims benefit of U.S. Provisional Application No. 60/501,613, filed Sep. 9, 2003, the disclosure of which is incorporated herein by reference.

### 2. FIELD OF THE INVENTION

This invention relates to diagnostic and/or therapeutic agents for use in ultrasound imaging incorporating ligands which have affinity for sites within the body.

### 3. BACKGROUND OF THE INVENTION

Apoptosis refers to “programmed cell death” whereby the cell executes a “cell suicide” program. It is now thought that the apoptosis program is evolutionarily conserved among virtually all multicellular organisms, as well as among all the cells in a particular organism. Further, it is believed that in many cases, apoptosis may be a “default” program that must be actively inhibited in healthy surviving cells. Apoptosis plays an important role in a number of physiological events including embryogenesis, regulation of the immune system, and homeostasis. Apoptosis also plays a role in the pathogenesis of a number of disorders including AIDS and other viral illnesses, cerebral ischemia, autoimmune and neurodegenerative diseases, organ and bone marrow transplant rejection, and tumor response to chemotherapy and radiation. Apoptosis can have particularly devastating consequences when it occurs pathologically in cells that do not normally regenerate, such as neurons. Because such cells are not replaced when they die, their loss can lead to debilitating and sometimes fatal dysfunction of the affected organ. Such dysfunction is evidenced in a number of neurodegenerative disorders that have been associated with increased apoptosis, including Alzheimer's disease, Parkinson's disease, amyotrophic lateral sclerosis, retinitis pigmentosa, hypoxic/ischemic injury in infants and adults, and cerebellar degeneration.

The consequences of undesired apoptosis can be similarly devastating in other pathologies as well, including ischemic injury, such as typically occurs in cases of myocardial infarction, reperfusion injury and stroke. In particular, apoptosis is believed to play a central role in very delayed infarction after mild focal ischemia (Du, 1996, J. Cereb. Blood Flow and Metab. 16:195-201). Apoptosis also plays a major role in coronary disease and atherosclerosis (Blankenberg and Strauss, 2001, Apoptosis 6:117-123). Monocytes are attracted to areas of endothelial damage where they infiltrate the arterial wall. As the area of damage is re-endothelialized, the new cells have increased permeability to circulating lipid and cholesterol complexes. This phenomena results in a collection of activated macrophages and lipid trapped below the endothelium, the birth of an atheroma. Apoptosis has been identified in the macrophages, smooth muscle cells, and in unstable plaque, of the endothelial cells forming the cap of the plaque. This last event is particularly dangerous as the apoptotic endothelial cells expressing PS on their surface (as discussed below) serve as thrombogenic foci, and may account for the presence of thrombus on atheroma with an intact cap.

One of the earliest events in apoptosis is the externalization of phosphatidylserine, a membrane phospholipid normally restricted to the inner leaflet of the lipid bilayer. Cells undergoing apoptosis redistribute phosphatidylserine from the inner leaflet of the plasma membrane lipid bilayer to the outer leaflet.

Annexins are a class of proteins that are characterized by calcium-mediated binding to anionic phospholipids. Annexin V is a human protein of 319 amino acids with a molecular weight of 36,000 Daltons and binds to phosphatidylserine with a high affinity (K_{d}=7 nmole/l). Because annexin has a high affinity for cell membranes expressing phosphatidylserine, annexin V derivatives have been utilized to detect apoptosis in hematopoietic cells, neurons, fibroblasts, endothelial cells, smooth muscle cells, carcinomas, lymphomas, all murine embryonic cell types and plant and insect cells. The utility of a radiolabeled annexin V for in vivo imaging of phosphatidylserine expression associated with apoptosis has been reported (U.S. Pat. Nos. 5,995,437, 6,171,577, 6,197,278 and 6,323,313). However, diagnostic techniques involving nuclear medicine generally involve exposure of the patient to ionizing electron radiation. Such radiation can cause damage to subcellular material, including deoxyribonucleic acid (DNA), ribonucleic acid (RNA) and proteins. In addition, such radiolabeled imaging agents persist for an extended period within the patient, often generate a high background signal, and are not entirely confined within the circulatory system.

There is a need to provide improved agents which would help in the early recognition of apoptosis. There is a need for improved annexin-based imaging agents that are effective for imaging apoptosis associated with vascular thrombi and with other pathologies. The present invention seeks to fulfill these needs and provides further related advantages.

### 4. SUMMARY OF THE INVENTION

In one aspect, the present invention provides annexin (e.g., annexin-V) coupled to ultrasound contrast particles for diagnosis and/or treatment of various pathological conditions characterized by apoptosis. The contrast particles can have a solid or hollow core, and can be single-layered, bi-layered or multi-layered. Annexin is coupled to the particles covalently or non-covalently to the particle surface. Reactive groups on the surface can be used, or reactive groups can be introduced onto the surface to enable attachment of annexin to the particles. The particles include a suitable ultrasound contrast agent, such as a gas, liquid or metal for ultrasound detection.

Another aspect of the invention concerns compositions and kits comprising annexin-coupled ultrasound particles. The particles can include a therapeutic agent.

In another aspect, the invention provides methods of diagnosing and/or imaging conditions characterized by apoptosis. The method generally involves administering a composition comprising annexin-coupled ultrasound contrast particles to a subject and obtaining an ultrasound image of at least a region of the subject. Because the annexin specifically binds to apoptotic cells and/or tissues, conditions characterized by apoptosis are revealed. In some embodiments, the composition is administered via intravenous injection. In some embodiments, unbound particles are permitted to clear from the circulatory system prior to imaging. The methods can be used to diagnose and/or image any condition characterized by apoptosis. As a specific example, the methods can be used as a non-radioactive, non-invasive means for diagnosing and/or monitoring for the presence of atherosclerotic plaques.

In an additional aspect, the invention involves methods in which annexin-coupled ultrasound contrast particles are used to convey therapeutic agents to the site of apoptosis, and in which the particles are subjected to ultrasound in order to release the agents.

The annexin-coupled particles, as described herein, can be used in ultrasound detection of tissues or cells undergoing apoptosis without the need for radiolabeled annexin, and therefore avoid radiation-induced damage associated with the use of radiolabeled compounds. The particles provide improved detection because the particles are confined within the circulatory system and because the particles are rapidly cleared by the reticulo-endothelial system (RES).

### 5. BRIEF DESCRIPTION OF THE DRAWINGS

Aspects of the invention can be more fully understood with respect to the following drawings:

FIG. 1 shows flow cytometry data demonstrating fluorescent avidin binding to biotinylated microparticles;

FIG. 2 shows flow cytometry data demonstrating fluorescent avidin binding to biotinylated microparticles after lyophilization and reconstitution;

FIG. 3 shows flow cytometry data demonstrating fluorescent avidin binding to microparticles reacted with hydrazide activated biotin;

FIG. 4 depicts the binding of avidin-coated microparticles to a microtitre well coated with biotinylated Protein A;

FIG. 5 depicts the binding of avidin-coated microparticles to a microtitre well coated with avidin;

FIG. 6 shows flow cytometry data demonstrating binding of IgG to Protein A coated microparticles; and

FIG. 7 shows flow cytometry data demonstrating fluorescently labeled biotin binding by antibody coupled to microparticles.

### 6. DETAILED DESCRIPTION OF THE PREFERRED EMBODIMENTS

6.1 Ultrasound Detection of Apoptosis

The present invention provides compositions, kits and methods that utilize annexin coupled to ultrasound contrast media for imaging, diagnosing and/or treating various conditions characterized by apoptosis, including, for example, coronary disease and atherosclerosis.

6.1.1 Ultrasound Imaging

One aspect of the present invention concerns the use of ultrasound imaging. Techniques for ultrasound imaging various parts of the body are well known. An ultrasonic scanner is placed on the body surface overlying the area to be imaged. The sound waves generated by the scanner are directed toward the area to be imaged. The scanner then detects sound waves reflected from the underlying area and translates the signal into images.

The acoustic properties of a substance, such as an organ system, will depend upon the velocity of the ultrasonic transmissions and the density of the substance. Changes in the substance's acoustic properties will be most prominent at the interface of components of the substance differing in density, such as solid, liquid, and gas components. When ultrasonic energy is transmitted through a substance, the changes in acoustic properties (e.g., density) of the substance cause changes in the reflection characteristics, resulting in a more intense sound reflection signal received by the ultrasonic scanner.

Ultrasound contrast agents are introduced into the body organ system being imaged using ultrasound, and there act to influence the ultrasound signal in a way that enhances the ultrasound image. The contrast agent may be ingested or injected into and perfuse the microvasculature of the tissue desired to be imaged, to provide clearer images of the tissue. Such ultrasound contrast agents may be useful in helping to diagnose diseases earlier and more accurately.

Liquid and solid contrast agents containing entrapped gas are known in the art. The gas microbubbles provided by these contrast agents act as sound wave reflectors because of the acoustic differences between the gas microbubble and surrounding liquid.

Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that can detect sound waves having a frequency of one megahertz (MHZ) to twenty MHZ. The detected waves can be integrated into an image which is quantitated and the quantitated waves converted into an image of the tissue being studied.

6.1.2 Ultrasound Contrast Particles and Contrast Agents

Another aspect of the invention concerns ultrasound contrast particles. The term “ultrasound contrast particle” (UCP) is defined herein to include essentially any conventional proteinaceous or polymeric micro- or nano-particle that can be used in ultrasound detection and to which annexin can be attached. The UCP can be solid or hollow-cored and can contain any suitable contrast agent such as a gas, liquid, metal. The outer surface of the UCP has, or can be modified to include, chemical features that permit charge and/or chemical modification, as described hereinbelow. It is not necessary for the UCP to be precisely spherical although they generally will be spherical and described as having average diameters. The UCP can be constructed such that the majority of those prepared in a composition will have diameters within the range of about one to ten microns in order to pass through the capillary system of the body. Alternatively, the UCP may be constructed with diameters below 1 micron, such as for instance in the range of 200 to 800 nm. The UCP can comprise a single layered shell or can comprise a shell with two or more layers encapsulating the core. The combined thickness of the outer and inner layers of the UCP shell will depend in part on the mechanical and drug carrying/delivering properties required of the microparticle, but typically the total shell thickness will be in the range of 10 to 750 nm.

The shell materials can include a wide variety of biodegradable and physiologically compatible materials, non-limiting examples of which include proteins such as albumin, gelatin, fibrinogen, and collagen as well as their derivatives, such as, succinylated gelatin, crosslinked polypeptides, reaction products of proteins with polyethylene glycol (e.g., albumin conjugated with polyethylene glycol), starch or starch derivatives, chitin, chitosan, pectin, biodegradable synthetic polymers such as polylactic acid, copolymers consisting of lactic acid and glycolic acid, polycyanoacrylates, polyesters, polyamides, polycarbonates, polyphosphazenes, polyamino acids, poly-ξ-caprolactone as well as copolymers consisting of lactic acid and ξ-caprolactone and their mixtures. Especially suitable are albumin, polylactic acid, copolymers consisting of lactic acid and glycolic acid, polycyanoacrylates, polyesters, polycarbonates, polyamino acids, poly-ξ-caprolactone as well as copolymers consisting of lactic acid, and ξ-caprolactone.

In one embodiment, the UCP is a single layered particle comprising a heat-denaturable biocompatible protein such as albumin as described in U.S. Pat. No. 4,957,656.

In another embodiment, the UCP is a polymeric spherical particle such as described in U.S. Pat. No. 6,544,496. In some embodiments, the polymer includes carbohydrates, carbohydrate derivatives and non-polyamino acid synthetic polymers. Examples of synthetic polymer include acrylates and polystyrenes. Examples of suitable carbohydrates include glucose, maltose, sucrose, sorbitol, dextrin, disaccharides, and polysaccharides.

Other UCPs useful in the present invention include those described in U.S. Pat. No. 5,543,158. The particles have a biodegradable solid core containing a biologically active material and poly(alkylene glycol) moieties on the surface. The terminal hydroxyl group of the poly(alkylene glycol) can be used to covalently attach onto the surface of the particles biologically active molecules. The surface of the particle can also be modified by attaching biodegradable polymers of the same structure as those forming the core of the particles. Examples of the biodegradable solid core include polyanhydride, polyhydroxybutyric acid, polyorthoesters, polysiloxanes, polycaprolactone, poly(lactic-co-glycolic acid), poly(lactic acid), poly(glycolic acid and copolymers prepared from the monomers of these polymers. The biodegradable moieties of the copolymer are in the core of the resulting particle, and the poly(alkylene glycol) moieties are on the surface of the resulting particle in an amount effective to decrease uptake of the particle by the reticuloendothelial system.

In another embodiment, the UCPs comprise particles as described in U.S. Pat. No. 6,007,845 in which non-linear multiblock copolymers are prepared by covalently linking a multifunctional compound with one or more hydrophilic polymers and one or more hydrophobic bioerodible polymers to form a polymer including at least three polymeric blocks. In one embodiment, one or more hydrophilic polymers, such as polyethylene glycol (PEG) chains or polysaccharide moieties, are covalently attached to a multifunctional molecule such as citric acid or tartaric acid, leaving one or more active hydroxyl, carboxylic acid or other reactive functional groups available to attach the hydrophobic polymer(s). The hydrophobic polymer, such as polylactic acid (PLA), polyglycolic acid (PGA), polyanhydrides, polyphosphazenes or polycaprolactone (PCL), is then covalently linked to the multifunctional compound via an appropriate reaction such as ring opening or condensation polymerization. The multifunctional compound can be selected from the group consisting of dextrins, pentaerythritol, glucaronic acid, tartaric acid, mucic acid, citric acid, benzene tricarboxylic acid, benzene tetracarboxylic acid and butane diglycidyl ether.

In other embodiments, the UCP is an ultrasmall porous particle such as described in U.S. Pat. No. 5,776,496. The pores can contain entrapped gas in the evacuated crevices or pores. In certain embodiments, the matrix material is iodipamide ethyl ester. Examples of suitable matrix material include iothalamate ethyl ester, iosefamate ethyl ester, 2,2′, 4 4′tetrahydroxybenzophenone, RS nitrocellulose, progesterone, beta-2,4,6-triiodo-3-dimethyl formamidinophenyl propionic acid ethyl ester, N-(trifluoroacetyl) Adrimycin 14 valerate, 1,2 diaminocyclohexane malinate platinum (ii), norethisterone, acetyl salicylic acid, warfarin, heparin-tridodecyl methyl ammonium chloride complex, sulfamethoxazole, cephalexin, prednisolone acetate, diazepam, clonazepam, methadone, naloxone, disulfiram, mercaptopurine, digitoxin, primaguine, mefloquine, atropine, scopolamine, thiazide, furosemide, propranolol, methyl methacrylate, poly methyl methacrylate, 5-fluorodeoxyuridine, cytosine arabinoside, acyclovir, levonorgestrel, aluminum chloride hexahydrate, the oxides of iron, copper, manganese, tin, mitindomide, isopropylpyrrolizine, iron citrate, iron iodate, calcium pyrophosphate, calcium salicylate, platinum dichloride and sodium pyrophosphate.

In other embodiments, the UCP is a microsphere formed from an amino acid polymer matrix with magnetic particles embedded therein such as described in U.S. Pat. Nos. 4,247,406 and 4,572,203.

In still other embodiments, the UCP is a microsphere comprising a carbohydrate polymer matrix such as described in U.S. Pat. Nos. 4,501,726, 4,713,249, and 4,687,748. Examples of suitable matrix materials include starch, dextran, gelatin, pullulan, alginate, chitosan, agarose, carrageenan, cellulose, glycogen, or a derivative thereof. In other embodiments, the matrix can be comprised of crystalline carbohydrate such as glucose, maltose, lactose, or carbohydrate polymer.

In yet another embodiment, the UCP is a bi-layered particle such as described in U.S. Pat. No. 6,193,951. In this embodiment, the inner layer of the shell is a biodegradable polymer, which may be a synthetic polymer, which may be tailored to provide the desired mechanical and acoustic properties to the shell or provide drug delivery properties. The outer layer of the shell is a biologically compatible material or biomaterial and is typically amphiphilic. The outer layer which is exposed to the blood and tissues serves as the biological interface between the particles and the body. The outer layer may also be formed of one or more synthetic biodegradable polymers. Materials useful for the outer shell include proteins such as collagen, gelatin or serum albumins, or globulins either derived from humans or having a structure similar to the human protein, glycosoaminoglycans such as hyaluronic acid, heparin and chondroitin sulphate and combinations or derivatives thereof. Synthetic biodegradable polymers, such as polyethylene glycol, polyethylene oxide, polypropylene glycol and combinations or derivatives may also be used. The inner shell can be a biodegradable polymer, which may be a synthetic polymer. Non-limiting examples are polycaprolactone, polylactide, polyglycolide, polyhydroxyvalerate, polyhydroxybutyrate, or copolymers thereof. A specific example is poly (D,L-lactide). The inner shell provides additional mechanical or drug delivery properties to the microparticle which are not provided or insufficiently provided by the outer layer, or enhances mechanical properties not sufficiently provided by the outer layer, without being constrained by surface property requirements. For example, a biocompatible outer layer of a cross-linked proteinaceous hydrogel can be physically supported using a high modulus synthetic polymer as the inner layer. The polymer may be selected for its modulus of elasticity and elongation, which define the desired mechanical properties. Typical biodegradable polymers include polycaprolactone, polylactic acid, polylactic-polyglycolic acid co-polymers, co-polymers of lactides and lactones, such as epsilon-caprolactone, δ-valerolactone, polyalkylcyanoacrylates, polyamides, polyhydroxybutryrates, polydioxanones, poly-β-aminoketones, polyanhydrides, poly-(ortho)esters, polyamino acids, such as polyglutamic and polyaspartic acids or esters of polyglutamic and polyaspartic acids. References on many biodegradable polymers are cited in Langer, et. al., 1983, Macromol. Chem. Phys. C23:61-125.

The UCPs can include at least one ultrasound contrast media. For example, the media can be a gas, liquid, or metal. Examples of liquid agents for ultrasound include emulsions and aqueous solutions (e.g., as described in U.S. Pat. No. 6,569,404). An example of a solid agent includes collagen microspheres. Gasses are commonly used as ultrasound media. The term “gas” as used herein includes any substance (including mixtures) substantially or completely in gaseous (including vapor) form at the normal human body temperature of 37° C. The gas or liquid is retained within the core of the particle. Non-limiting examples of suitable gases include air; nitrogen; oxygen; carbon dioxide; hydrogen; nitric oxide; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as methylsilane or dimethylsilane; a low molecular weight hydrocarbon (e.g., containing up to 7 carbon atoms), for example an alkane such as methane, ethane, a propane, a butane or a pentane, a cycloalkane such as cyclopropane, cyclobutane or cyclopentane, an alkene such as ethylene, propene, propadiene or a butene, or an alkyne such as acetylene or propyne; an ether such as dimethyl ether; a ketone; an ester; a halogenated low molecular weight hydrocarbon (e.g., containing up to 7 carbon atoms); or a mixture of any of the foregoing. At least some of the halogen atoms in halogenated gases are fluorine atoms; thus biocompatible halogenated hydrocarbon gases may, for example, be selected from bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethylfluoride, 1,1-difluoroethane and perfluorocarbons, e.g., perfluoroalkanes such as perfluoromethane, perfluoroethane, perfluoropropanes, perfluorobutanes (e.g., perfluoro-n-butane, optionally in admixture with other isomers such as perfluoro-iso-butane), perfluoropentanes, perfluorohexanes and perfluoroheptanes; perfluoroalkenes such as perfluoropropene, perfluorobutenes (e.g., perfluorobut-2-ene) and perfluorobutadiene; perfluoroalkynes such as perfluorobut-2-yne; and perfluorocycloalkanes such as perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane and perfluorocycloheptane. Other halogenated gases include methyl chloride, fluorinated (e.g., perfluorinated) ketones such as perfluoroacetone and fluorinated (e.g., perfluorinated) ethers such as perfluorodiethyl ether. Perfluorinated gases, for example sulphur hexafluoride and perfluorocarbons such as perfluoropropane, perfluorobutanes and perfluoropentanes, may be used. In some embodiments, the gas may comprise a substance such as butane, cyclobutane, n-pentane, isopentane, neopentane, cyclopentane, perfluoropentane, perfluorocyclopentane, perfluorohexane or a mixture containing one or more such gases which is liquid at handling or processing temperatures but gaseous at body temperature. Other examples of ultrasound image enhancing materials include metals such as Fe, Al, Ni, Co, Cu, Ag, Mn, and Pt as described in U.S. Pat. No. 4,687,748. Additional examples include glass, graphite, metal flakes, mangenite, magnetic iron oxide, and carbonyl iron as described in U.S. Pat. No. 4,572,203.

The UCP shell can be subjected to stabilizing procedures. In one embodiment, the UCP includes a proteinaceous outer shell which has been subjected to a cross-linking procedure, such as treatment with glutaraldehyde or carbodiimide. In another example, the UCP comprises a proteinaceous shell whose exterior surfaces have been stabilized by treatment with metal (e.g., chromium) salts to form polynulcear coordination complexes between the ionized carboxyl groups of the surface-exposed amino acids and bridging ligands (e.g., as described in U.S. Pat. No. 6,083,484). Such treatments are useful in giving the UCPs sufficient mechanical strength so that they may also be sized or processed after manufacture, such as described in U.S. Pat. No. 6,193,951. The UCPs can also be subjected to surface modifying methods and agents that may be used to passivate against macrophages and/or the reticuloendothelial system (RES) such as described in U.S. Pat. No. 6,193,951 and co-pending U.S. application Ser. No. 09/637,516. The UCPs of the present invention preferably are substantially free from film-forming surfactants (e.g., phospholipids) (such as described in U.S. Pat. No. 6,264,917).

The UCP can include within its core a substance, such as a therapeutic agent, to be delivered. Non-limiting examples of such an agent include peptides, proteins, carbohydrates, nucleic acids, lipids, polysaccharides, combinations thereof, and synthetic inorganic or organic molecules that cause a biological effect when administered to an animal. Other examples of such therapeutic agents include drugs to limit ischemic injury to the heart, a drug to limit reperfusion injury to the heart, or a drug to limit restenosis of a coronary artery. Specific examples include cardiovascular drugs (endocardium agents) with short circulatory half-lives that affect the cardiac tissues, vasculature and endothelium to protect and treat the hear from ischemic or reperfusion injury or coronary artery from restenosis (anti-restenosis agent). Drugs which target platelets (anti-platelet agent) and white cells (anti-white cell agent) which may plug the microvasculature of the heart after a heart attack are also useful for local cardiac delivery. Another type of drug useful for local delivery is one for which a local effect is required but where systemic effects of the drug would be detrimental. These are typically drugs with high toxicity, for example, locally administered potent vasodilators which would increase blood flow to hypoxic tissue but if delivered systemically would cause a dangerous drop in blood pressure. Suitable drugs include fibrinolytic agents such as tissue plasminogen activator, streptokinase, urokinase, and their derivatives, vasodilators such as verapamil, multifunctional agents such as adenosine, adenosine agonists, adenosine monophosphate, adenosine diphosphate, adenosine triphosphate, and their derivatives, white cell or platelet acting agents such as GPIIb/IIIa antagonists, energy conserving agents such as calcium channel blockers, magnesium and beta blockers, endothelium acting agents such as nitric oxide, nitric oxide donors, nitrates, and their derivatives, free-radical scavenging agents, agents which affect ventricular remodeling such as ACE inhibitors and agiongenic agents, and agents that limit restenosis of coronary arteries after balloon angioplasty or stenting. The use of ultrasound contrast agents serving also as drug carriers has been described in WO/03034975 and in U.S. Pat. Nos. 5,190,766, 5,580,575 and 6,284,280.

The wall thickness, mechanical properties and porosity of the one or more layers in the shell of UCPs may be adjusted by varying the concentration of components during manufacture by conventional methods (e.g., as described in U.S. Pat. No. 6,193,951 and WO/03034975). In particular, by appropriately adjusting the mechanical properties, the particles may be made to remain stable to threshold diagnostic imaging power, while being rupturable by an increase in power and/or by being exposed to its resonant frequency. The resonant frequency can be made to be within the range of transmitted frequencies of diagnostic body imaging systems or can be a harmonic of such frequencies. In some embodiments, UCP compositions may be made having a resonant frequency greater or equal to 2 MHz, and typically greater or equal to 5 MHz. In one embodiment, the UCPs can be manufactured to have a controlled fragility characterized by a uniform wall thickness to diameter ratio that defines a discrete threshold power intensity value of ultrasonic energy where UCP rupture occurs.

6.2 Apoptosis

Another aspect of the invention concerns the diagnosis and/or treatment of conditions associated with, or characterized by, apoptosis. It is generally believed that biological membranes are asymmetric with respect to specific membrane phospholipids. In particular, the outer leaflet of eukaryotic plasma membranes is formed predominantly with the cholinephospholipids, such as sphingomyelin and phosphatidylcholine (PC), whereas the inner leaflet contains predominantly aminophospholipids, such as phosphatidylserine (PS) and phosphatidylethanolamine (PE). This asymmetry is thought to be maintained by the activity of an adeno sine triphosphate (ATP)-dependent aminophospho lipid translocase, which selectively transports PS and PE between bilayer leaflets (Seigneuret and Devaux, 1984, Proc. National Acad. Sci. USA 81:3751). Other enzymes thought to be involved in the transport of phospholipids between leaflets include ATP-dependent floppase (Connor, et al., 1992, J. Biol. Chem. 267:19412) and lipid scramblase (Zwaal, et al., 1993, Biochem. Soc. trans. 21:248).

Although asymmetry appears to be the rule for normal cells, the loss of such asymmetry is associated with certain physiological, as well as pathogenic, processes. For example, it has been recognized that membrane asymmetry, detected as appearance of PS on the outer leaflet of the plasma membrane (“PS exposure”), is one of the earliest manifestations of apoptosis, preceding DNA fragmentation, plasma membrane blebbing, and loss of membrane integrity (Martin, et al., 1995, J. Exp. Med. 182:1545; Fadok, et al., 1992, J. of Immunol. 149:4029).

6.2.1 Annexin

An additional aspect of the invention concerns the use of annexin for targeting cells and tissues undergoing apoptosis. Annexin is normally found in high levels in the cytoplasm of a number of cells including placenta, lymphocyte, monocytes, biliary and renal (cortical) tubular epithelium. Although the physiological function of annexins has not been fully elucidated, several properties of annexins make them useful as diagnostic and/or therapeutic agents. In particular, it has been discovered that annexins possess a very high affinity for anionic phospholipid surfaces, such as a membrane leaflet having an exposed surface of phosphatidylserine (PS). As used herein, the term “annexin” refers to a class of proteins characterized by their ability to bind with high affinity to membrane lipids in the presence of millimolar concentrations of calcium. Annexins have been shown to exhibit anticoagulatory effects that are mediated by the binding of annexins to negatively charged surface phospholipids (e.g., on activated platelets). Annexin V is a representative annexin molecule used in the description of the present invention. Annexins within the scope of the invention include annexins I, II, III, IV, V, VI, VII, VIII, XI, XIII, XXXI, and XXXII. The term “annexin” includes native annexin purified from natural sources such as, for example, human placenta, or annexin molecules containing a native sequence produced through genetic engineering, recombinant, or other means. Purified annexin V is commercially available (e.g., from Sigma-Aldrich, product no. A9640), and labeled (e.g., biotinylated) preparations are also available (e.g., from BD-Biosciences and Sigma-Aldrich). The term “annexin” also includes modified annexins derived from or produced by any source. As used herein, the term “modified annexin” refers to an annexin molecule wherein the native sequence or molecule is altered in such a way without materially altering the membrane binding affinity of the annexin. A number of different annexins have been cloned from humans and other organisms. Their sequences are available in sequence databases, including GenBank. Such annexins can be produced by chemical, genetic engineering, or recombinant techniques. The modification can include sequence modification through the addition and/or deletion of several amino acid residues, and/or an addition/deletion of an amino acid at a single site on the native or genetically engineered sequence. Amino acid replacements can be conservative replacements. In some embodiments, amino acids that are readily modifiable (e.g., cysteine or serine) are introduced. Examples of modified annexins include annexins modified at the N-terminus by the addition of amino acid residues including cysteine (U.S. Pat. No. 6,323,313) which can be used to covalently attach UCP as described below.

In certain embodiments, the invention involves the use of annexin V. Annexin V is one of the most abundant annexins, (ii) it is simple to produce from natural or recombinant sources, and (iii) it has a high affinity for phospholipid membranes (Tait, et al., 1988, Biochemistry 27:6268). Human annexin V has a molecular weight of 36 kd and high affinity (K_{d}=7 nmol/l) for phosphatidylserine (PS). The sequence of human annexin V can be obtained from GenBank under accession numbers U05760-U05770.

An exemplary expression system suitable for making annexin employs the pET12a-PAPI expression vector (Novagen, Madison, Wis.) in E. coli (Wood, 1996, Blood 88:1873-1880). Other bacterial expression vectors can be utilized as well. They include, e.g., the plasmid pGEX (Smith, et al., 1988, Gene 67:31) and its derivatives (e.g., the pGEX series from Pharmacia Biotech, Piscataway, N.J.). These vectors express the polypeptide sequences of a cloned insert fused in-frame with glutathione-S-transferase. Recombinant pGEX plasmids can be transformed into appropriate strains of E. coli and fusion protein production can be induced by the addition of IPTG (isopropyl-thio galactopyranoside). Solubilized recombinant fusion protein can then be purified from cell lysates of the induced cultures using glutathione agarose affinity chromatography according to standard methods (Ausubel, et al., 1995, Current Protocols in Molecular Biology, John Wiley and Sons, Inc., Media, Pa.). Other commercially-available expression systems include yeast expression systems, such as the Pichia expression kit from Invitrogen (San Diego, Calif.); baculovirus expression systems (Reilly, et al., 1992, Baculovirus Expression Vectors: A laboratory Manual; Beames, et al., 1991, Biotechniques 11:378); Clontech, Palo Alto Calif.); and mammalian cell expression systems (Clontech, Palo Alto Calif.; Gibco-BRL, Gaithersburg Md.).

A number of features can be engineered into the expression vectors, such as leader sequences which promote the secretion of the expressed sequences into culture medium. The recombinantly produced polypeptides are typically isolated from lysed cells or culture media. Isolated recombinant polypeptides produced as described above may be purified by standard protein purification procedures, including differential precipitation, molecular sieve chromatography, ion-exchange chromatography, isoelectric focusing, gel electrophoresis and affinity chromatography. Protein preparations can also be concentrated by, for example, filtration (Amicon, Danvers, Mass.).

6.3 Compositions and Kits Containing Annexin Coupled to Ultrasound Contrast Particles

In another aspect, the invention concerns UCPs that have been coupled to annexin. Coupling of a UCP to an annexin may be achieved by any conventional means that does not interfere with the ability of the UCP to provide ultrasound contrast and that does not interfere with the binding properties of annexin. For example, a linking agent can be used to couple a UCP to annexin. The identity of the linking agent is not a critical aspect of the present invention. Any conventional linking agent can be used so long as it does not interfere with the function of UCP-annexin conjugate. The linking agent can be used to form a linkage of any combination of atoms that will function to couple the UCP to annexin, and can be used to make either a chemical linkage of one or more atoms, or a bond without linkage atoms. The identity of the linking agent will depend upon the nature of the desired conjugation. For example, the coupling may be mediated by covalent attachment, in which case the linking agent includes reactive moieties that are either capable of forming covalent linkages with complementary functional groups or are capable of being activated so as to form covalent linkage with complementary functional groups; or mediated through the use of pairs of specific binding molecules, such as biotin and avidin/streptavidin.

In some embodiments, the linking agent comprises two or more reactive moieties connected by a spacer element. The presence of such a spacer permits bifunctional linkers to react with specific functional groups within a molecule or between two different molecules, resulting in a bond between these two components and introducing extrinsic linker-derived material into the UCP-annexin conjugate. The reactive moieties in a linking agent may be the same (homobifunctional agents) or different (heterobifunctional agents or, where several dissimilar reactive moieties are present, heteromultifunctional agents), providing a diversity of potential reagents that may bring about covalent bonding between any chemical species, either intramolecularly or intermolecularly. Examples of chemically reactive functional groups which may be employed in conjunction with bifunctional linkers include amino, hydroxyl, sulfhydryl, carboxyl, and carbonyl groups, as well as carbohydrate groups, vicinal diols, thioethers, 2-aminoalcohols, 2-aminothiols, guanidinyl, imidazolyl and phenolic groups. Examples of reactive moieties capable of reaction with sulfhydryl groups include α-haloacetyl compounds of the type X—CH₂CO— (where X═Br, Cl or I), which show particular reactivity for sulfhydryl groups but which can also be used to modify imidazolyl, thioether, phenol and amino groups as described by Gurd, 1967, in Methods Enzymol. 11:532. N-Maleimide derivatives are considered selective towards sulfhydryl groups, but may additionally be useful in coupling to amino groups under certain conditions. In specific examples, a homo-bifunctional maleimide cross-linker could attach a free sulfhydryl group in annexin to a free sulfhydryl group on the surface of a UCP. Or, a hetero-bifunctional maleimide/hydrazide linker could attach a free sulfhydryl group in annexin to a free aldehyde group on the surface of a UCP. Reagents which introduce reactive disulphide bonds into either the UCP or annexin may be useful, since linking may be brought about by disulphide exchange between annexin and the UCP; examples of such reagents include Ellman's reagent (DTNB), 4,4′-dithiodipyridine, methyl-3-nitro-2-pyridyl disulphide and methyl-2-pyridyl disulphide (described by Kimura, T. et al., 1982, Analyt. Biochem. 122:271).

Non-limiting examples of heterobifunctional linking agents include malemide-hydrazide heterobifunctional cross-linkers. In one embodiment, these agents are used to attach annexin having a free sulfhydryl group to a free aldehyde on the surface of a UCP. Aldehydes can be introduced into the proteinaceous shell of a UCP by the use of glutaraldehyde cross-linking agent. The following are non-limiting examples of such heterobifunctional linking agents: KMUH, (N-[k-maleimidoundecanoic acid]hydrazide) (Pierce catalog no. 22111); EMCH, ([N-e-maleimidocaproic acid]hydrazide) (Pierce catalog no. 22106); BMPH, [N-(β-maleimidopropionic acid) hydrazide (trifluoroacetic acid salt)] (Pierce catalog no. 22297); and MPBH, 4-(4-N-maleimidophenyl)butyric acid hydrazide hydrochloride (Pierce catalog no. 22305).

Non-limiting examples of reactive moieties capable of reaction with amino groups include alkylating and acylating agents. Representative alkylating agents include:
i) α-haloacetyl compounds, which show specificity towards amino groups in the absence of reactive thiol groups and are of the type X—CH₂O-(where X═Cl, Br or I), e.g., as described by Wong, Y-H. H., 1979. Biochemistry 24:5337;
ii) N-maleimide derivatives, which may react with amino groups either through a Michael type reaction or through acylation by addition to the ring carbonyl group as described by Smyth, D. G. et al., 1960, J. Am. Chem. Soc. 82:4600 and Smyth, D. G. et al., 1964, Biochem. J. 91:589;
iii) aryl halides such as reactive nitrohaloaromatic compounds;
iv) alkyl halides as described by McKenzie, J. A. et al., 1988, J. Protein Chem. 7:581;
v) aldehydes and ketones capable of Schiff's base formation with amino groups, the adducts formed usually being stabilized through reduction to give a stable amine;
vi) epoxide derivatives such as epichlorohydrin and bisoxiranes, which may react with amino, sulfhydryl or phenolic hydroxyl groups;
vii) chlorine-containing derivatives of s-triazines, which are very reactive towards nucleophiles such as amino, sulfhydryl and hydroxy groups;
viii) aziridines based on s-triazine compounds detailed above, e.g., as described by Ross, W. C. J., 1954, Adv. Cancer Res. 2:1, which react with nucleophiles such as amino groups by ring opening;
ix) squaric acid diethyl esters as described by Tietze, L. F., 1991, Chem. Ber. 124:1215; and
x) α-haloalkyl ethers, which are more reactive alkylating agents than normal alkyl halides because of the activation caused by the ether oxygen atom, e.g., as described by Benneche, T. et al., 1993, Eur. J. Med. Chem. 28:463.

Representative amino-reactive acylating agents include:
i) isocyanates and isothiocyanates, particularly aromatic derivatives, which form stable urea and thiourea derivatives respectively and have been used for protein crosslinking as described by Schick, A. F. et al., 1961, J. Biol. Chem. 236:2477;
ii) sulfonyl chlorides, which have been described by Herzig, D. J. et al., 1964, Biopolymers 2:349;
iii) Acid halides;
iv) Active esters such as nitrophenylesters or N-hydroxysuccinimidyl esters;
v) acid anhydrides such as mixed, symmetrical or N-carboxyanhydrides;
vi) other useful reagents for amide bond formation as described by Bodansky, M. et al., 1984, in “Principles of Peptide Synthesis” Springer-Verlag;
vii) acylazides, e.g., wherein the azide group is generated from a preformed hydrazide derivative using sodium nitrite, e.g., as described by Wetz, K. et al., 1974, Anal. Biochem. 58:347;
viii) azlactones attached to polymers such as bis-acrylamide, e.g., as described by Rasmussen, J. K., 1991, Reactive Polymers 16:199; and
ix) Imidoesters, which form stable amidines on reaction with amino groups, e.g., as described by Hunter, M. J. and Ludwig, M. L., 1962, J. Am. Chem. Soc. 84:3491.

Carbonyl groups such as aldehyde functions may be reacted with weak protein bases at a pH such that nucleophilic protein side-chain functions are protonated. Weak bases include 1,2-aminothiols such as those found in N-terminal cysteine residues, which selectively form stable 5-membered thiazolidine rings with aldehyde groups, e.g., as described by Ratner, S. et al., 1937, J. Am. Chem. Soc. 59:200. Other weak bases such as phenyl hydrazones may be used, e.g., as described by Heitzman, H. et al., 1974, Proc. Natl. Acad. Sci. USA 71:3537.

Aldehydes and ketones may also be reacted with amines to form Schiff's bases, which may advantageously be stabilized through reductive amination. Alkoxylamino moieties readily react with ketones and aldehydes to produce stable alkoxyamines, e.g., as described by Webb, R. et al., 1990, Bioconjugate Chem. 1:96.

Examples of reactive moieties capable of reaction with carboxyl groups include diazo compounds such as diazoacetate esters and diazoacetamides, which react with high specificity to generate ester groups, e.g., as described by Herriot R. M., 1947, Adv. Protein Chem. 3:169. Carboxylic acid modifying reagents such as carbodiimides, which react through O-acylurea formation followed by amide bond formation, may also usefully be employed; linking may be facilitated through addition of an amine or may result in direct annexin-UCP coupling. Useful water soluble carbodiimides include 1-cyclohexyl-3-(2-morpholinyl-4-ethyl)carbodiimide (CMC) and 1-ethyl-3-(3-dimethylaminopropyl)carbodiimide (EDC), e.g., as described by Zot, H. G. and Puett, D., 1989, J. Biol. Chem. 264:15552. Other useful carboxylic acid modifying reagents include isoxazolium derivatives such as Woodwards reagent K; chloroformates such as p-nitrophenylchloroformate; carbonyldiimidazoles such as 1,1′-carbonyldiimidazole; and N-carbalkoxydihydroquinolines such as N-(ethoxycarbonyl)-2-ethoxy-1,2-dihydroquinoline.

Other potentially useful reactive moieties include vicinal diones such as p-phenylenediglyoxal, which may be used to react with guanidinyl groups, e.g., as described by Wagner et al., 1978, Nucleic acid Res. 5:4065; and diazonium salts, which may undergo electrophilic substitution reactions, e.g., as described by Ishizaka, K. and Ishizaka T., 1960, J. Immunol. 85:163. Bis-diazonium compounds are readily prepared by treatment of aryl diamines with sodium nitrite in acidic solutions. It will be appreciated that functional groups in the UCP and/or annexin may if desired be converted to other functional groups prior to reaction, e.g., to confer additional reactivity or selectivity. Examples of methods useful for this purpose include conversion of amines to carboxylic acids using reagents such as dicarboxylic anhydrides; conversion of amines to thiols using reagents such as N-acetylhomocysteine thiolactone, S-acetylmercaptosuccinic anhydride, 2-iminothiolane or thiol-containing succinimidyl derivatives; conversion of thiols to carboxylic acids using reagents such as α-haloacetates; conversion of thiols to amines using reagents such as ethylenimine or 2-bromoethylamine; conversion of carboxylic acids to amines using reagents such as carbodiimides followed by diamines; and conversion of alcohols to thiols using reagents such as tosyl chloride followed by transesterification with thioacetate and hydrolysis to the thiol with sodium acetate.

Annexin-UCP coupling may also be effected using enzymes as zero-length linking agents; thus, for example, transglutaminase, peroxidase and xanthine oxidase may be used to produce linked products. Reverse proteolysis may also be used for linking through amide bond formation.

Non-covalent annexin-UCP coupling may, for example, be effected by electrostatic charge interactions e.g., between a polylysinyl-functionalized UCP and a polyglutamyl-functionalized annexin, through chelation in the form of stable metal complexes or through high affinity binding interaction such as avidin/biotin or avidin/streptavidin binding.

It is also possible to obtain molecules that bind specifically to the surface of UCPs by direct screening of molecular libraries for UCP-binding molecules. For example, phage libraries displaying small peptides may be used for such selection. The selection may be made by simply mixing the UCPs and the phage display library and eluting the phages binding to the floating UCPs. If desired, the selection may be done under “physiological conditions” (e.g., in blood) to eliminate peptides which cross-react with blood components. An advantage of this type of selection procedure is that only binding molecules that do not destabilize the UCPs should be selected, since only binding molecules attached to intact floating UCPs will rise to the top. It may also be possible to introduce some kind of “stress” during the selection procedure (e.g., pressure) to ensure that destabilizing binding moieties are not selected. Furthermore the selection may be done under shear conditions, for example by first letting the phages react with the UCPs and then letting the UCPs pass through a surface coated with anti-phage antibodies under flow conditions. In this way it may be possible to select binders which may resist shear conditions present in vivo.

Coupling may also be effected using avidin or streptavidin, which have four high affinity binding sites for biotin. Avidin may be used to conjugate annexin to UCP if both annexin and UCP are biotinylated. Details of such a conjugation are provided in the Examples hereinbelow. This method may also be extended to include linking of UCP to UCP, a process which may encourage UCP association and consequent potentially increased echogenicity. Alternatively, avidin or streptavidin may be attached directly to the surface of UCPs.

Non-covalent coupling may also utilize the bifunctional nature of bispecific immunoglobulins. These molecules can specifically bind two antigens, thus linking them. For example, either bispecific IgG or chemically engineered bispecific F(ab)′₂ fragments may be used as linking agents. Heterobifunctional bispecific antibodies have also been reported for linking two different antigens, e.g., as described by Bode, C. et al., 1989, J. Biol. Chem. 264:944 and by Staerz, U. D. et al., 1986, Proc. Natl. Acad. Sci. USA 83:1453. A UCP and annexin may be crosslinked by antibody molecules and lead to formation of multi-UCP cross-linked assemblies of potentially increased echogenicity.

Linking agents used in accordance with the invention will in general bring about linking of annexin to UCP or UCP to UCP with some degree of specificity, and may also be used to attach one or more therapeutically active agents.

So-called zero-length linking agents, which induce direct covalent joining of two reactive chemical groups without introducing additional linking material (e.g., as in amide bond formation induced using carbodiimides or enzymatically) may, if desired, be used in certain embodiments.

In some embodiments, no bifunctional linking agent is required to effect a coupling between a UCP and a protein ligand (e.g., annexin, or an antibody). Applicants observed, as described hereinbelow in relation to FIG. 7, that microparticles having a cross-linked albumin outer layer could be coupled directly to a protein ligand (an IgG) without the use of a heterobifunctional cross-linker. Without wishing to be bound by theory, this coupling may be due either to the non-covalent association of the IgG with the glutaraldehyde cross-linked albumin on the microparticle's surface or by the covalent attachment of the ligand to the surface of the microparticle via the formation of a Schiff's base between the free aldehydes on the surface of the microparticle and free amines on the IgG.

The nature of extrinsic material introduced by the linking agent, as discussed above, may have a bearing on the targeting ability and general stability of the ultimate product. Thus, it may be desirable to introduce labile linkages, e.g., containing spacer arms which are biodegradable or chemically sensitive or which incorporate enzymatic cleavage sites. Alternatively the spacer may include polymeric components, e.g., to enhance UCP stability. The spacer may also contain reactive moieties to enhance surface crosslinking, or it may contain a tracer element such as a fluorescent probe, spin label or radioactive material.

The surface density of bound annexin can be modulated and will affect the in vivo binding properties. For example, higher surface density can be used to enhance detection sensitivity, which can enable binding to lower density targets.

Spacer elements may typically consist of aliphatic chains which effectively separate the reactive moieties of the linker by distances of between 5 and 30 Å. They may also comprise macromolecular structures such as poly(ethylene glycol) (PEG) (see, e.g., Milton Harris, J. (ed) “Poly(ethylene glycol) chemistry, biotechnical and biomedical applications” Plenum Press, New York, 1992). PEGs are soluble in most solvents, including water, and are highly hydrated in aqueous environments, with two or three water molecules bound to each ethylene glycol segment; this has the effect of preventing adsorption either of other polymers or of proteins onto PEG-modified surfaces. PEGs are known to be nontoxic and not to harm active proteins or cells, whilst covalently linked PEGs are known to be non-immunogenic and non-antigenic. Furthermore, PEGs may readily be modified and bound to other molecules with only little effect on their chemistry. Their advantageous solubility and biological properties are apparent from the many possible uses of PEGs and copolymers thereof, including block copolymers such as PEG-polyurethanes and PEG-polypropylenes. Appropriate molecular weights for PEG spacers used in accordance with the invention may, for example, be between 120 Daltons and 20 kDaltons.

Other representative spacer elements include structural-type polysaccharides such as polygalacturonic acid, glycosaminoglycans, heparinoids, cellulose and marine polysaccharides such as alginates, chitosans and carrageenans; storage-type polysaccharides such as starch, glycogen, dextran and aminodextrans; polyamino acids and methyl and ethyl esters thereof, as in homo- and co-polymers of lysine, glutamic acid and aspartic acid; and polypeptides, oligosaccharides and oligonucleotides, which may or may not contain enzyme cleavage sites.

In various embodiments, spacer elements may contain cleavable groups such as vicinal glycol, azo, sulfone, ester, thioester or disulphide groups. Spacers may contain biodegradable methylene diester or diamide groups of formula

-(Z)ₘYXC(R¹R²)XY(Z)ₙ—

[where X and Z are selected from —O—, —S—, and —NR— (where R is hydrogen or an organic group); each Y is a carbonyl, thiocarbonyl, sulphonyl, phosphoryl or similar acid-forming group: m and n are each zero or 1; and R¹ and R² are each hydrogen, an organic group or a group —XY(Z)ₘ-, or together form a divalent organic group] may also be useful; as discussed in, for example, WO-A-9217436 such groups are readily biodegraded in the presence of esterases, e.g., in vivo, but are stable in the absence of such enzymes. They may therefore advantageously be linked to therapeutic agents to permit slow release thereof.

Poly[N-(2-hydroxyethyl)methacrylamides] are potentially useful spacer materials by virtue of their low degree of interaction with cells and tissues (see, e.g., Volfova, I., Rihova, B. and V. R. and Vetvicka, P., 1992, J. Bioact. Comp. Polymers 7:175-190).

Other potentially useful polymeric spacer materials include:
i) copolymers of methyl methacrylate with methacrylic acid; these may be erodible (see Lee, P. I., 1993, Pharm. Res. 10:980) and the carboxylate substituents may cause a higher degree of swelling than with neutral polymers;
ii) block copolymers of polymethacrylates with biodegradable polyesters (see, e.g., San Roman, J. and Guillen-Garcia, P., 1991, Biomaterials 12:236-241);
ii) cyanoacrylates, i.e. polymers of esters of 2-cyanoacrylic acid; these are biodegradable and have been used in the form of nanoparticles for selective drug delivery (see Forestier, F., Gerrier, P., Chaumard, C., Quero, A. M., Couvreur, P. and Labarre, C., 1992, J. Antimicrob. Chemoter. 30:173-179);
iv) polyvinyl alcohols, which are water-soluble and generally regarded as biocompatible (see, e.g., Langer, R., 1991, J. Control. Release 16:53-60);
v) copolymers of vinyl methyl ether with maleic anhydride, which have been stated to be bioerodible (see Finne, U., Hannus, M. and Urtti, A., 1992, Int. J. Pharm. 78:237-241);
vi) polyvinylpyrrolidones, e.g., with molecular weight less than about 25,000, which are rapidly filtered by the kidneys (see Hespe, W., Meier, A. M. and Blankwater, Y. M., 1977, Arzeim.-Forsch./Drug Res. 27:1158-1162);
vii) polymers and copolymers of short-chain aliphatic hydroxyacids such as glycolic, lactic, butyric, valeric and caproic acids (see, e.g., Carli, F., 1993, Chim. Ind. (Milan) 75:494-9), including copolymers which incorporate aromatic hydroxyacids in order to increase their degradation rate (see Imasaki, K., Yoshida, M., Fukuzaki, H., Asano, M., Kumakura, M., Mashimo, T., Yamanaka, H. and Nagai. T., 1992, Int. J. Pharm. 81:31-38);
viii) polyesters consisting of alternating units of ethylene glycol and terephthalic acid, e.g., Dacron®, which are non-degradable but highly biocompatible;
ix) block copolymers comprising biodegradable segments of aliphatic hydroxyacid polymers (see, e.g., Younes, H., Nataf, P. R., Cohn, D., Appelbaum, Y. J., Pizov, G. and Uretzky, G., 1988, Biomater. Artif. Cells Artif. Organs 16:705-719), for instance in conjunction with polyurethanes (see Kobayashi, H., Hyon, S. H. and Ikada, Y., 1991, “Water-curable and biodegradable prepolymers” J. Biomed. Mater. Res. 25:1481-1494);
x) polyurethanes, which are known to be well-tolerated in implants, and which may be combined with flexible “soft” segments, e.g., comprising poly(tetra methylene glycol), poly(propylene glycol) or poly(ethylene glycol) and aromatic “hard” segments, e.g., comprising 4,4′-methylenebis(phenylene isocyanate) (see, e.g., Ratner, B. D., Johnston, A. B. and Lenk, T. J., 1987, J. Biomed. Mater. Res: Applied Biomaterials 21:59-90; Sa Da Costa, V. et al., 1981, J. Coll. Interface Sci. 80:445-452 and Affrossman, S. et al., 1991, Clinical Materials 8:25-31);
xi) poly(1,4-dioxan-2-ones), which may be regarded as biodegradable esters in view of their hydrolysable ester linkages (see, e.g., Song, C. X., Cui, X. M. and Schindler, A., 1993, Med. Biol. Eng. Comput. 31:S 147-150), and which may include glycolide units to improve their absorbability (see Bezwada, R. S., Shalaby, S. W. and Newman, H. D. J., 1990, Agricultural and synthetic polymers: Biodegradability and utilization (ed Glass, J. E. and Swift, G.), 167-174 ACS symposium Series, #433, Washington D.C., U.S.A., American Chemical Society);
xii) polyanhydrides such as copolymers of sebacic acid (octanedioic acid) with bis(4-carboxy-phenoxy)propane, which have been shown in rabbit studies (see Brem, H., Kader, A., Epstein, J. I., Tamargo, R. J., Domb, A., Langer, R. and Leong, K. W., 1989, Sel. Cancer Ther. 5:55-65) and rat studies (see Tamargo, R. J., Epstein, J. I., Reinhard, C. S., Chasin, M. and Brem, H., 1989, J. Biomed. Mater. Res. 23:253-266) to be useful for controlled release of drugs in the brain without evident toxic effects;
xiii) biodegradable polymers containing ortho-ester groups, which have been employed for controlled release in vivo (see Maa, Y. F. and Heller, J., 1990, J. Control. Release 14:21-28); and
xiv) polyphosphazenes, which are inorganic polymers consisting of alternate phosphorus and nitrogen atoms (see Crommen, J. H., Vandorpe, J. and Schacht, E. H., 1993, J. Control. Release 24:167-180).

The following tables list linking agents and protein modification agents which may be useful in preparing annexin-UCP conjugates in accordance with embodiments of the invention.

Heterobifunctional Linking Agents

| Linking agent | Reactivity 1 | Reactivity 2 | Comments |
|---|---|---|---|
| KMUH | —SH | aldehyde | |
| EMCH | —SH | aldehyde | |
| BMPH | —SH | aldehyde | |
| MPBH | —SH | aldehyde | |
| ABH | carbohydrate | photoreactive | |
| ANB-NOS | —NH₂ | photoreactive | |
| APDP(1) | —SH | photoreactive | iodinable |
| | | | disulphide |
| | | | linker |
| APG | —NH₂ | photoreactive | reacts |
| | | | selectively |
| | | | with Arg at pH |
| | | | 7-8 |
| ASIB(1) | —SH | photoreactive | iodinable |
| ASBA(1) | —COOH | photoreactive | iodinable |
| EDC | —NH₂ | —COOH | zero-length |
| | | | linker |
| GMBS | —NH₂ | —SH | |
| sulfo-GMBS | —NH₂ | —SH | water-soluble |
| HSAB | —NH₂ | photoreactive | |
| sulfo-HSAB | —NH₂ | photoreactive | water-soluble |
| MBS | —NH₂ | —SH | |
| sulfo-MBS | —NH₂ | —SH | water-soluble |
| M₂C₂H | carbohydrate | —SH | |
| NHS-ASA(1) | —NH₂ | photoreactive | iodinable |
| sulfo-NHS- | —NH₂ | photoreactive | water-soluble, |
| ASA(1) | | | iodinable |
| sulfo-NHS-LC- | —NH₂ | photoreactive | water-soluble, |
| ASA(1) | | | iodinable |
| PDPH | carbohydrate | —SH | disulphide |
| | | | linker |
| PNP-DTP | —NH₂ | photoreactive | |
| SADP | —NH₂ | photoreactive | disulphide |
| | | | linker |
| sulfo-SADP | —NH₂ | photoreactive | water-soluble |
| | | | disulphide |
| | | | linker |
| SAED | —NH₂ | photoreactive | disulphide |
| | | | linker |
| SAND | —NH₂ | photoreactive | water-soluble |
| | | | disulphide |
| | | | linker |
| SANPAH | —NH₂ | photoreactive | |
| sulfo-SANPAH | —NH₂ | photoreactive | water-soluble |
| SASD(1) | —NH₂ | photoreactive | water-soluble |
| | | | iodinable |
| | | | disulphide |
| | | | linker |
| SIAB | —NH₂ | —SH | |
| sulfo-SIAB | —NH₂ | —SH | water-soluble |
| SMCC | —NH₂ | —SH | |
| sulfo-SMCC | —NH₂ | —SH | water-soluble |
| SMPB | —NH₂ | —SH | |
| sulfo-SMPB | —NH₂ | —SH | water-soluble |
| SMPT | —NH₂ | —SH | |
| sulfo-LC-SMPT | —NH₂ | —SH | water-soluble |
| SPDP | —NH₂ | —SH | |
| sulfo-SPDP | —NH₂ | —SH | water-soluble |
| sulfo-LC-SPDP | —NH₂ | —SH | water-soluble |
| sulfo-SAMCA(2) | —NH₂ | photoreactive | |
| sulfo-SAPB | —NH₂ | photoreactive | water-soluble |

Notes:
(1)= iodinable; (2)= fluorescent

Homobifunctional Linking Agents

| Linking agent | Reactivity | Comments |
|---|---|---|
| BS | —NH₂ | |
| BMH | —SH | |
| BASED(1) | photoreactive | iodinable disulphide linker |
| BSCOES | —NH₂ | |
| sulfo-BSCOES | —NH₂ | water-soluble |
| DFDNB | —NH₂ | |
| DMA | —NH₂ | |
| DMP | —NH₂ | |
| DMS | —NH₂ | |
| DPDPB | —SH | disulphide linker |
| DSG | —NH₂ | |
| DSP | —NH₂ | disulphide linker |
| DSS | —NH₂ | |
| DST | —NH₂ | |
| sulfo-DST | —NH₂ | water-soluble |
| DTBP | —NH₂ | disulphide linker |
| DTSSP | —NH₂ | disulphide linker |
| EGS | —NH₂ | |
| sulfo-EGS | —NH₂ | water-soluble |
| SPBP | —NH₂ | |

Biotinylation Agents

| Agent | Reactivity | Comments |
|---|---|---|
| biotin-BMCC | —SH | |
| biotin-HPDP | —SH | disulphide linker |
| biotin-hydrazide | carbohydrate | |
| biotin-LC-hydrazide | carbohydrate | |
| iodoacetyl-LC-biotin | —NH₂ | |
| NHS-iminobiotin | —NH₂ | reduced affinity for |
| | | avidin |
| NHS-SS-biotin | —NH₂ | disulphide linker |
| photoactivatable biotin | nucleic | |
| | acids | |
| sulfo-NHS-biotin | —NH₂ | water-soluble |
| sulfo-NHS-LC-biotin | —NH₂ | |

Notes:
DPPE = dipalmitoylphosphatidylethanolamine; LC = long chain

Agents for Protein Modification

| Agent | Reactivity | Function |
|---|---|---|
| Ellman's reagent | —SH | quantifies/detects/protects |
| DTT | —SS— | reduction |
| 2-mercaptoethanol | —SS— | reduction |
| 2-mercaptylamine | —SS— | reduction |
| Traut's reagent | —NH₂ | introduces —SH |
| SATA | —NH₂ | introduces protected —SH |
| AMCA-NHS | —NH₂ | fluorescent labeling |
| AMCA-hydrazide | carbohydrate | fluorescent labeling |
| AMCA-HPDP | —SS— | fluorescent labeling |
| SBF-chloride | —SS— | fluorescent detection of —SH |
| N-ethylmaleimide | —SS— | blocks —SH |
| NHS-acetate | —NH₂ | blocks and acetylates —NH₂ |
| citraconic anhydride | —NH₂ | reversibly blocks and |
| | | introduces negative charges |
| DTPA | —NH₂ | introduces chelator |
| BNPS-skatole | tryptophan | cleaves tryptophan residue |
| Bolton-Hunter | —NH₂ | introduces iodinable group |

6.4 Formulations of Annexin-UCPs

Once prepared, UCPs coupled to annexin can be freeze-dried for storage using conventional methods such as described in U.S. Pat. Nos. 6,165,442 and 6,193,951. The final formulation of the UCPs after preparation, but prior to use, is in the form of a lyophilized cake. The later reconstitution of the UCPs may be facilitated by lyophilization with bulking agents which provide a cake having a high porosity and surface area.

Bulking agents added during or before lyophilization of the particles may be used to control the osmolality of the final formulation for injection. An osmolality other than physiological osmolality may be desirable during lyophilization to minimize aggregation. When the particles are reformulated for injection, the volume of liquid used to reconstitute the particles must be properly calculated to compensate, if necessary to provide an injectable composition with a physiologically acceptable osmolal balance. The bulking agents may also increase the drying rate during lyophilization by providing channels for the water and solvent vapor to be removed. This also provides a higher surface area which would assist in the later reconstitution. Typical bulking agents are sugars such as dextrose, mannitol, sorbitol and sucrose, and polymers such as PEG's and PVP's. Other additives may be included in the particle suspension prior to lyophilization in order to prevent aggregation or to facilitate dispersion of the UCPs upon reconstitution. Surfactants, such as poloxamers (polyethylene glycol-polypropylene glycol-polyethylene glycol block co-polymers), may be used as well as water soluble polymers, such as medium molecular weight polyethyleneglycols and low to medium molecular weight polyvinylpyrrolidones, which may assist in the dispersion of the UCP. In one embodiment, the freeze-dried preparation includes a water soluble polymer at a weight percentage in the range of approximately 30 to 70; a surfactant at a weight percentage in the range of approximately 8 to 15%; and an osmolality adjusting agent at a weight percentage in the range of approximately 20 to 30%.

If the formulation is to contain a drug-containing core, the annexin-UCP may be soaked in a solution of the drug whereby the solution diffuses into the interior. In particular, the use of bilayered UCPs where the inner shell has a porous characteristic allows for rapid diffusion of a drug solution into the hollow core. The UCPs may be re-dried such as by lyophilization to produce a gas filled, drug containing UCP. The combination of the drug with prefabricated particles allows one to avoid processing which may lead to drug degradation. To provide UCPs having a solid core containing a drug, during formation of the UCPs, the thickness of the inner layers may be increased to occupy more or all of the interior volume. Then, by later soaking in the drug-containing solution, the inner solid core will absorb the drug and provide a solid reservoir for the drug. Alternatively, the drug may be dissolved in the organic phase with the biopolymer during the UCP forming process. Evaporation of the organic solvents causes the drug to co-precipitate with the biopolymer inside the UCP.

The production of the annexin-UCP suspension is typically carried out by resuspending freeze-dried UCPs in a pharmaceutically compatible suspension medium. The resuspending in a suitable medium can be tied directly to the last process step (the freeze-drying), but can optionally also be carried out by the attending physician just before administration. In the latter case, the UCPs according to the invention can be present as a kit that consists of a first container that contains the UCP coupled to annexin, and a second container that contains the suspension medium. The size of the first container is to be selected in such a way that the latter also has plenty of room for the suspension medium. Thus, e.g., with the aid of a syringe via a membrane located in the seal of the first container, the suspension medium can be added completely to the annexin-UCP, and the suspension that is ready for injection can be produced by subsequent shaking. As suspension media, all injectable media that are known to one skilled in the art, such as, e.g., physiological common salt solution, water p.i. or 5% glucose solution, are suitable. The amount administered depends on the active ingredient enclosed in each case. As a rough upper limit, a value can be assumed, as would also be used in conventional administration of the respective active ingredient. Because of the action-enhancing effect as well as the possibility of releasing the active ingredient specifically from the UCP-annexin conjugates according to the invention, the necessary dose, however, generally lies below this upper limit.

The compositions of the invention can be formulated into diagnostic compositions for enteral or parenteral administration. These compositions contain an effective amount of the annexin-UCP along with conventional pharmaceutical carriers and excipients appropriate for the type of administration contemplated. Parenteral compositions may be injected directly or mixed with a large volume parenteral composition for systemic administration. Such solutions also may contain pharmaceutically acceptable buffers and, optionally, electrolytes such as sodium chloride. Formulations for enteral administration may vary widely, as is well-known in the art. In general, such formulations are liquids which include an effective amount of the annexin-UCP in aqueous solution or suspension. Such enteral compositions may optionally include buffers, surfactants, thixotropic agents, and the like. Compositions for oral administration may also contain flavoring agents and other ingredients for enhancing their organoleptic qualities.

The annexin-UCP are administered in doses effective to achieve the desired enhancement of the ultrasound image. Such doses may vary widely, depending upon the particular agent employed, the organs or tissues which are the subject of the imaging procedure, the imaging procedure, the imaging equipment being used, and the like, and will be apparent to those of skill in the art.

6.5 Utility of Annexin-UCP Conjugate

The annexin-UCP of the invention can be used in the conventional manner. The compositions may be administered to a patient, typically a warm-blooded animal, either systemically, for example by intravenous injection, or locally to the organ or tissue to be imaged, and the patient then subjected to the imaging procedure. Protocols for imaging and instrument procedures are readily found in texts. In some embodiments, for example embodiments in which the background concentration of the annexin-UPC particles would obscure visualization of the target tissue or organ, it may be desirable to wait a period of time sufficient for unbound particles to clear the circulatory system prior to imaging. For example, in applications designed to diagnose and/or image atherosclerotic plaques and/or other prothrombotic conditions, it may be desirable to wait a period of time sufficient for the unbound annexin-UPCs to clear the circulatory system before imaging via ultrasound.

Therapeutics may easily be delivered in accordance with the invention to diseased or necrotic areas, for example in the heart, general vasculature, and to the liver, spleen, kidneys and other regions such as the lymph system, body cavities or gastrointestinal system. Products according to the present invention may be used for targeted therapeutic delivery either in vivo or in vitro.

In another aspect, the invention concerns methods of imaging apoptosis in a region of interest in a mammalian subject in vivo. In one embodiment, the method can include: administering to the subject a composition comprising a plurality of UCPs coupled to annexin; positioning an ultrasound probe in relation to said subject in order to measure back scattering ultrasound energy; measuring back scattering ultrasound energy from the subject to construct an image of ultrasound contrast; wherein the image is a representation of cells undergoing apoptosis in said region. The method can include repetition of the measuring at selected intervals in order to track changes in the intensity of radiation emission from the region over time, reflecting changes in the number of cells undergoing apoptosis. In some embodiments, for example embodiments in which the composition is administered via intravenous injection, the unbound particles are permitted to clear from the circulatory system by waiting a sufficient period of time prior to imaging.

By means of the present invention it is possible to recognize the prothrombotic state of the vascular system. This diagnosis is made possible by the specificity of the annexins of the present invention which are capable of recognizing the prothrombotic state of any cell of interest (e.g., platelet or endothelial cell), which is different from the normal state. Since the prothrombotic state differs from the normal state of the cell in that the outer coat of only the prothrombotic cell shows phosphatidyl serine, this principle can be exploited according to the invention by any agent capable of specifically distinguishing phosphatidyl serine from phosphatidyl choline. The agents which may be used according to the invention are characterized by their specificity for phosphatidyl serine, which can be determined by the binding tests described in the specification. By making use of this specificity of the annexin-UCP according to the invention it is possible to locate the starting point for the activation of the hemostatic system and/or the thrombus. Consequently, the present invention provides agents which make it possible to initiate suitable therapeutic measures by providing for the early diagnosis of a state which might possibly develop into a health-threatening condition. The agents can also be used to monitor the efficacy of treatments by comparing images obtained prior to initiating therapy with those taken during and/or after a specified treatment regimen.

The invention having been described, various features and advantages of the invention are illustrated in the following examples, which are intended to be illustrative and non-limiting.

### 7. EXAMPLES

### 7.1 Example 1

### Covalent Attachment of Biotin to the Surface of Bilayered Microparticles Via Endogenous Free Sulfhydryl Groups

A prelyophilization suspension of glutaraldehyde cross-linked, human albumin-coated, cyclooctane-filled microparticles was produced essentially as described in Example 11 of U.S. Pat. No. 6,193,951. The resulting microparticles were retrieved by centrifugation and washed 2 times in an aqueous solution of 0.25% poloxamer 188 (Spectrum Chemicals and Laboratory Products, Gardena, Calif., Product #P1169).

Each human serum albumin molecule contains 35 cysteines. The sulfhydryl groups of 34 of theses cysteines participate in the formation of the 17 disulfide bonds which are essential to the protein's three dimensional conformation, while the sulfhydryl group of one cysteine remains available for directed modification. Biotin was covalently attached to the microparticle surface using a biotin-containing reagent capable of reacting with free sulfhydryl groups as described below.

The microparticles were concentrated by centrifugation at about 3000×g and resuspended in 10 mM phosphate buffered saline, 150 mM NaCl, 2 mM EDTA, pH 7.4 (PBSE) at a particle density of approximately 2×10⁹/ml and an albumin concentration of approximately 1 mg/ml. PEO-maleimide activated biotin (PMAB, Pierce Chemical, Product # 21901) was dissolved in PBSE and added to aliquots of the resuspended microparticles at final concentrations of 0 (untreated control), 0.038, 0.19, 1.9 mM PMAB and incubated at room temperature with gentle agitation for 3 hours. The control and PMAB-treated microparticles were concentrated by centrifugation, washed and resuspended in 0.25% poloxamer 188.

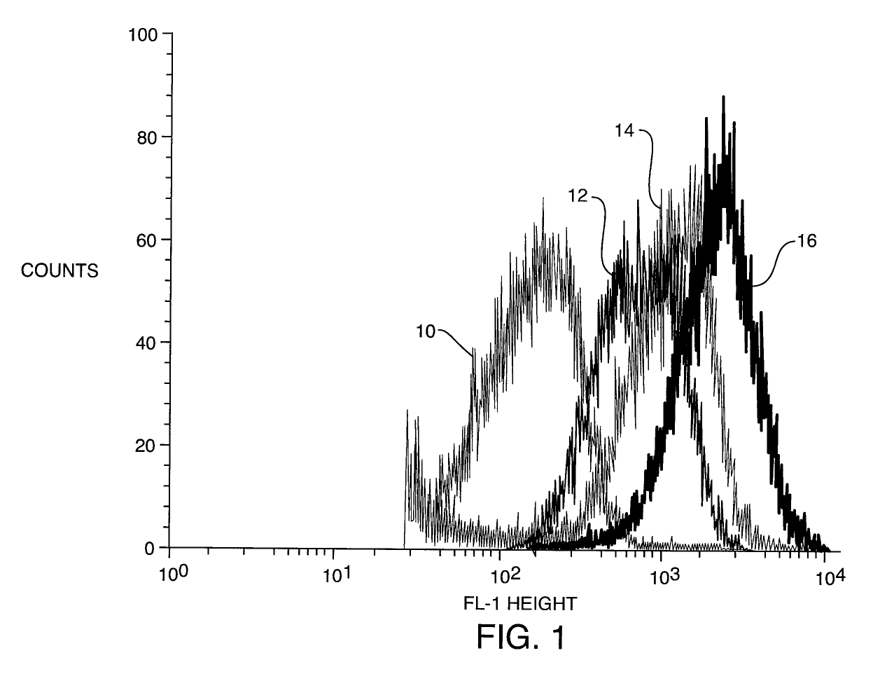

Aliquots of the washed microparticles were resuspended in PBSE and incubated with fluorescently labeled avidin (F-Av, Pierce Chemical, Product # 21221) with gentle agitation for 30 minutes prior to the detection of fluorescently labeled avidin on the surface of the particles using a flow cytometer (BD FacsCalibur). Avidin, a protein isolated from hen egg whites, rapidly binds noncovalently to biotin with the highest reported affinity for any protein ligand pair (Kₐ=10¹⁵M⁻¹). Increasing amounts of F-Av bind to the PMAB-treated microparticles in a manner which is proportional to the concentration of PMAB used to treat the microparticles (FIG. 1 wherein ref. nos. 10, 12, 14, and 16 refer to PMAB concentrations of 0, 0.038, 0.19, and 1.9 mM, respectively). These results indicate that the surface of the microparticles has been modified by the attachment of biotin and that the biotin attached to the surface retains its capacity to bind to avidin.

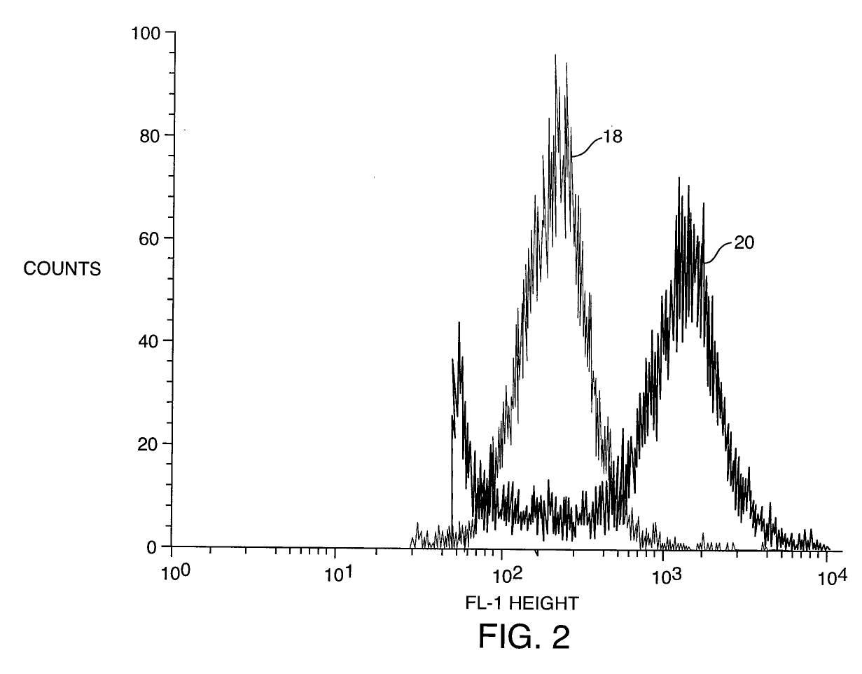

Aliquots of the washed microparticles treated with 1.9 mM PMAB were formulated with appropriate excipients, dispensed to vials and lyophilized as previously described (U.S. Pat. No. 6,193,951). Lyophilized microparticles were reconstituted with DI water and aliquots were then incubated with F-Av prior to analysis with the flow cytometer. FIG. 2 demonstrates that the avidin binding capacity of the biotinylated microparticles has been retained in the lyophilized, reconstituted microparticles (ref. nos. 18 and 20 refer to particles prepared with PMAB concentrations of 0 and 1.19 mM, respectively).

### 7.2 Example 2

### Covalent Attachment of Biotin to the Surface of Bilayered Microparticles Via Residual Aldehyde Groups

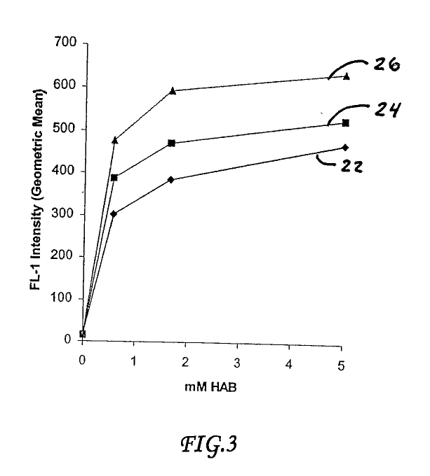

Biotin was coupled to the surface of glutaraldehyde cross-linked, human albumin-coated, cyclooctane-filled microparticles in a manner similar to that described in Example 1 with the exception that a hydrazide activated biotin capable of reacting with free carbonyl groups (HAB, Pierce Chemical, Product #21340) was used instead of PMAB. In contrast to the PMAB biotinylation of the microparticles which utilizes the endogenous sulfhydryl groups present in the human albumin, biotinylation with HAB utilizes the residual carbonyl (aldehyde) groups which are the result of the glutaraldehyde cross-linking that stabilizes the albumin surface during the process of manufacturing the microparticles. Time and concentration dependent biotinylation of the surface of the microparticles was demonstrated by flow cytometry after incubation with F-Av as in Example 1 (FIG. 3 in which the ref. nos. 22, 24, 26 refer to HAB reaction times of 2 hr, 6 hr, and 24 hr, respectively).

### 7.3 Example 3

### Covalent Attachment of PEG to the Surface of Bilayer Microparticles Via DTT Generated SH Groups

The cross-linked albumin surface of the microparticles can be further altered using a variety of techniques to provide additional reactive groups for surface modification. For example, some of the 34 sulfhydryl groups that are chemically unavailable for modification due to their participation in the 17 disulfide bonds present in the albumin molecule can be converted to free sulfhydryls by treatment with reducing agents, such as dithiothreitol (DTT), thus creating additional opportunities on the particle surface for modification with sulfhydryl directed reagents.

In this example, some of the disulfide bonds present on the surface of a prelyophilization suspension of glutaraldehyde cross-linked, human albumin-coated, cyclooctane-filled microparticles were reduced by treatment with 20 mM DTT in 50 mM Tris-Cl, 5 mM EDTA, pH 8.6. After the reduced microparticles were concentrated and separated from DTT they were resuspended in PBSE+0.25% poloxamer 188. Aliquots were incubated at room temperature with gentle agitation for 69 hours with a maleimide activated PEG at 0 (reduced, unmodified control), 2.67, or 13.3 mg/ml. (Shearwater Chemicals, Product #2D2M0H01). The control and PEG-treated microparticles were purified and concentrated by centrifugation. The free sulfhydryl content of aliquots of the reduced microparticles, as well as the reoxidized control and the PEG-treated microparticles was assessed spectrophotometrically at 412 nm after reaction with the sulfhydryl reactive reagent DTNB (Sigma Prod. #D-8130) using an extinction coefficient of 14150 M⁻¹ cm⁻¹.

Protein concentration of the samples was determined by BCA assay (Pierce Chemical, Product #23227). The extent of PEG incorporation was determined by the additional decrease in free sulfhydryl groups in the PEG-treated samples relative to the reduced, reoxidized control. As seen in the following Table, approximately 6-8 of the 22 free sulfhydryl groups/albumin that were generated by the original DTT reduction were subsequently modified by the addition of the sulfhydryl directed PEGs in a concentration dependent manner.

### Incorporation of Maleimide Activated PEGs at DTT Generated Sulfhydryl Groups on the Surface of Microparticles

| | Molar ratio SH: | Molar ratio PEG: |
|---|---|---|
| SAMPLE | albumin | albumin |
| reduced microparticle | 21.8 | 0 |
| reduced reoxidized control | 12.0 | 0 |
| maleimide PEG treated | 6.3 | 5.7 |
| (2.67 mg/ml) | | |
| maleimide PEG treated | 3.6 | 8.4 |
| (13.3 mg/ml) | | |

### 7.4 Example 4

### Production of Avidin-Coated Bilayered Microparticles

The surface of biotinylated microparticles produced as in Example 1 were stably coated with avidin by dilution into a stirred solution of avidin (Sigma A-9275, 1 mg/ml in PBS). Avidin-coated microparticles were concentrated and purified from free avidin by centrifugation and washing with 0.25% poloxamer 188 prior to formulation with appropriate excipients and lyophilization.

Each molecule of avidin has the capacity to bind 4 molecules of biotin. Thus, coating biotinylated microparticles with avidin should produce particles which are now capable of binding to subsequently added biotin or biotinylated molecules. Avidin's tetravalent biotin binding, in combination with its high affinity for biotin, has led to the widespread use of avidin/biotin reagents in biomedical diagnostic and research applications.

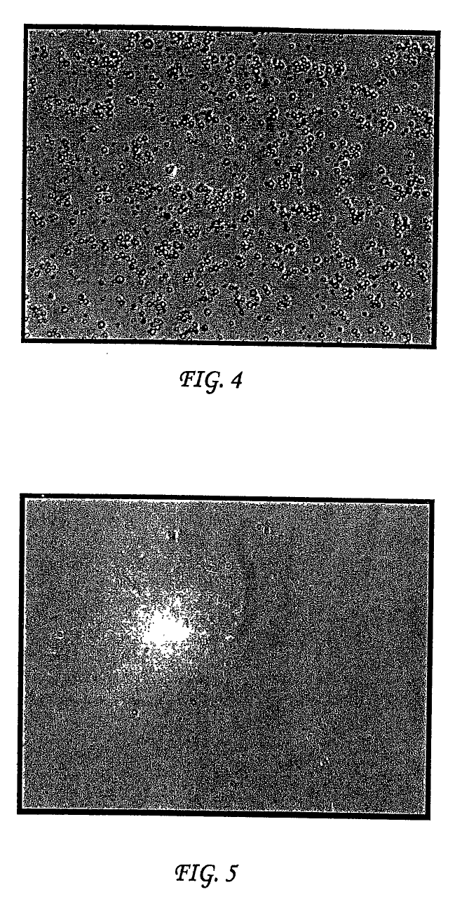

Avidination of the biotinylated microparticles and lyophilized, reconstituted microparticles was demonstrated on the flow cytometer after incubation with fluorescently labeled biotin (Sigma Product B-9431). In addition, the biotin binding capacity of avidin-coated microparticles was demonstrated by microscopic examination of their immobilization in a microtitre well which was previously coated with a biotinylated target protein, protein A (Sigma Product P-2165) (FIG. 4). These same particles failed to bind to a microtitre well coated with avidin, further demonstrating that the biotinylated particles had been uniformly covered with avidin (FIG. 5).

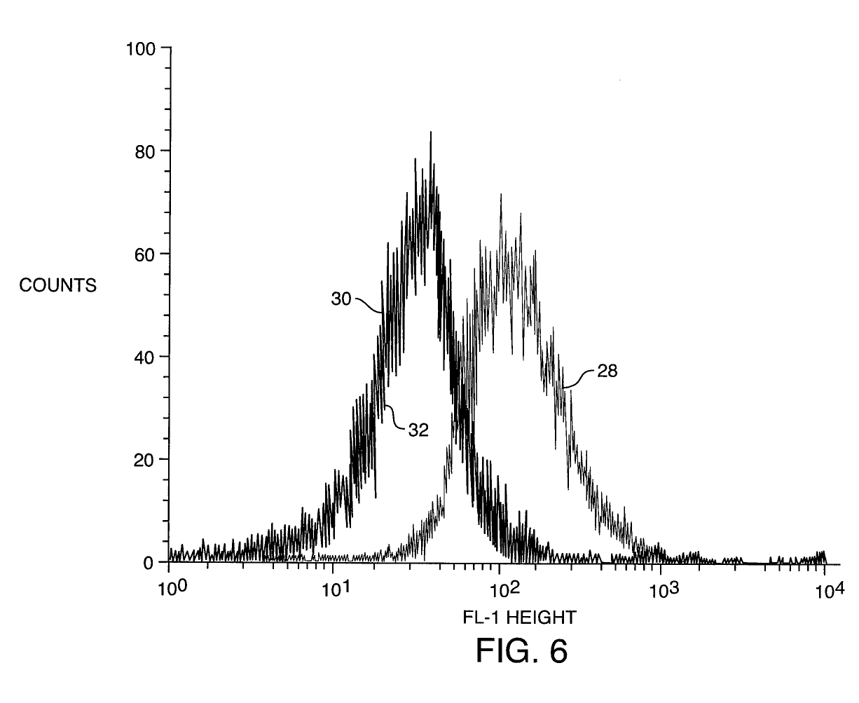

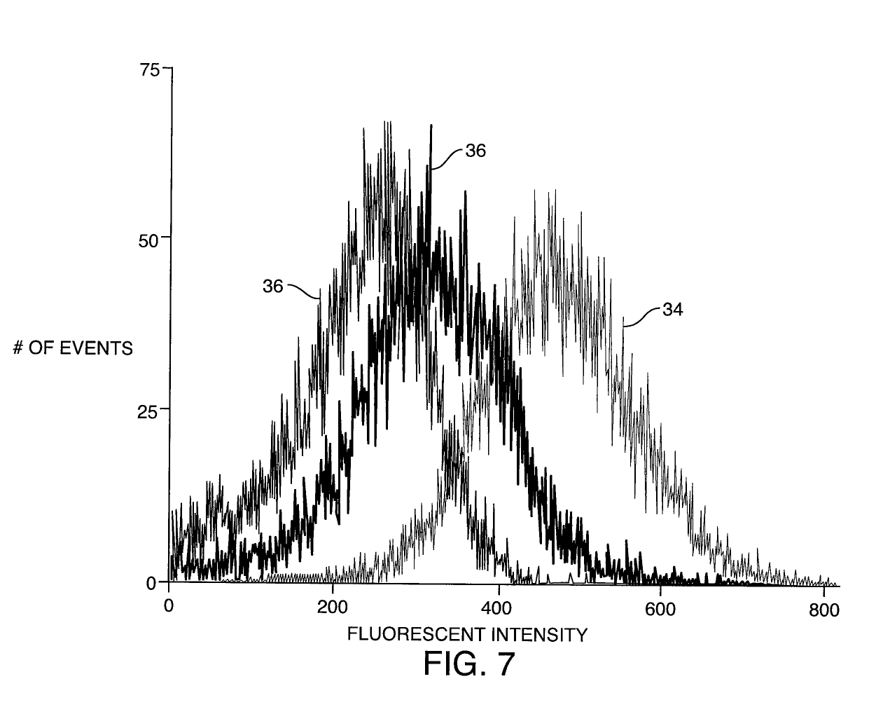

Maintenance of functional binding activity of biotinylated Protein A immobilized on the surface of avidin-coated particles was also demonstrated in solution by incubating avidin-coated particles with biotinylated Protein A in order to coat the particles with Protein A. Protein A, isolated from S. aureus has the capacity to bind a variety of mammalian immunoglobulins. Functional binding activity of the Protein A-coated microparticles was evaluated using flow cytometry to demonstrate the selective immobilization of a fluorescently labeled monoclonal IgG antibody (BD Biosciences, Palo Alto, Calif., Product #347497) on the surface of Protein A-coated microparticles (FIG. 6). The fluorescent antibody was immobilized on the surface of aviding coated microparticles which were previously coated with biotinylated Protein A (28). The fluorescent antibody was not immobilized on the surface of avidin coated microparticles which were not previously coated with biotinylated Protein A (30) or on biotinylated microparticles previously incubated with biotinylated Protein A (32).

### 7.5 Example 5

### Binding Annexin-V-Coated Microparticles to Apoptotic Cells In Vivo

Annexin-V is a 320 amino acid protein which displays calcium dependent, high affinity binding to phosphatidyl serine. Phosphatidyl serine, a component of the membrane of mammalian cells, is normally present only on the interior surface of the cell membrane but appears on the surface of cells which are in the process of apoptosis. Thus, annexin-V will bind selectively to cells which have begun apoptosis. Identification of regions of apoptosis may be useful for a variety of conditions including the diagnosis and treatment of atherosclerotic disease where increased apoptosis may be an indicator of the impending rupture of atherosclerotic plaque and the onset of acute myocardial infarction or in evaluating the efficacy of cancer treatments which induce apoptosis in malignant cells.

Annexin-V was immobilized on the surface of microparticles by incubating biotinylated annexin-V with reconstituted avidin-coated particles produced as in Example 4. The capacity of the annexin-coated microparticles to bind to apoptotic cells in vivo was then demonstrated by ultrasound enhancement in a rabbit model of atherosclerosis (induced focal vascular apoptosis) after intravenous administration of the annexin-coated microparticles. Rabbits, fed a high fat diet to induce atherosclerosis, and presumed to have severe atherosclerotic disease in their aorta, were used in this study. A baseline ultrasound image was first taken of abdominal aorta using an Acuson Sequoia with a 15L8 Transducer. The annexin-V labeled microparticle suspension was intravenously injected. Thirty minutes after injection, the same region of aorta was again imaged by ultrasound. No circulating microparticles were observed and an increase in brightness of the lumen of the aorta was clearly visible in a region corresponding to the expected area of atherosclerosis (data not shown). Histopathologic analysis of the imaged region of aorta confirmed the presence of apoptotic cells.

### 7.6 Example 6

### Covalent Attachment of Antibody to the Surface of Microparticles with a Maleimide-Hydrazide Heterobifunctional Cross-Linker

KMUH(N-[k-maleimidoundecanoic acid]hydrazide), a heterobifunctional cross-linker, was used to couple a polyclonal antibody to the microparticle surface, making use of the reaction of the cross-linker's hydrazide group with pre-existing aldehydes on the microparticle, and the reaction of its maleimide group with a free sulfhydryl on the antibody. Since antibody molecules typically do not contain free sulfhydryl groups, one was added to the antibody used herein through the use of Traut's reagent (2-iminothiolane) which will react with free amino groups present in the antibody resulting in the introduction of free sulfhydryl groups. Specifically, 1 mg/ml goat anti-biotin IgG (Sigma #B3640) was reacted at room temperature for 75 min. with 8 μg/ml 2-iminothiolane (Sigma #16256) in phosphate buffered saline, pH 8, 2 mM EDTA. The thiolated antibody was separated from free 2-iminothiolane by size exclusion chromatography on a Baker G-25 SPE desalting mini-column (7219-07) which was pre-equilibrated and eluted with phosphate buffered saline, pH 7, 2 mM EDTA. KMUH (Pierce #22111) was dissolved in DMSO at 10 mg/ml and added to the thiolated antibody (0.5 mg/ml) to a final concentration of 0.3 mg/ml and allowed to react at room temperature for 2 hours. The KMUH-activated antibody was separated from free KMUH by size exclusion chromatography on a Baker G-25 SPE desalting mini-column which was pre-equilibrated and eluted with 0.2 M sodium acetate buffer, pH 5.5. The KMUH-activated goat anti-biotin IgG was mixed with an equal volume of glutaraldehyde cross-linked, human albumin-coated, cyclooctane-filled microparticles suspended in 0.25% poloxamer 188 at a particle density of approximately 2×10⁹/ml and an albumin concentration of approximately 1 mg/ml. The coupling reaction was allowed to proceed at room temperature for 12 hours with gentle agitation. The microparticles were concentrated by centrifugation, washed and resuspended in 0.25% poloxamer 188. The immobilization of goat anti-biotin IgG on the surface of the microparticles was demonstrated on the flow cytometer after incubation with fluorescently labeled biotin (Sigma Product B-9431) (FIG. 7). Increased antigen binding is seen with the microparticles treated with the KMUH-activated goat anti-biotin (34) compared to either untreated microparticles (36) or microparticles which were treated with goat anti-biotin antibody without prior activation (38). The increased antigen binding seen in FIG. 7 with the antibody treated microparticles (38) relative to the untreated microparticles (36) indicates that the microparticles can also be coated directly with this antibody without the use of the KMUH heterobifunctional cross-linker. Without wishing to be bound by theory, this coating may be due either to the non-covalent association of antibody with the glutaraldehyde cross-linked albumin on the microparticle's surface or by the covalent attachment of the antibody to the surface via the formation of a Schiff's base between the free aldehydes on the surface of the microparticles and free amines in antibody.

While the foregoing has presented specific embodiments of the present invention, it is to be understood that these embodiments have been presented by way of example, only. It is expected that others will perceive and practice variations which, though differing from the foregoing do not depart from the spirit and scope of the invention as described and claimed herein. All patent applications, patents, and literature references cited in this specification are hereby incorporated by reference in their entirety.

1. An ultrasound reagent useful for imaging apoptosis, comprising: a polymeric particle capable of reflecting sound waves and an annexin V, wherein the annexin V is substantially permanently linked to the particle.

2. The reagent of claim 1 in which the polymeric particle is a matrix particle comprising a polymer having a contrast agent capable of reflecting sound waves entrapped therein.

3. The reagent of claim 2 in which the polymer is selected from the group consisting of a polypeptide, a non-synthetic biodegradable polymer, a synthetic biodegradable polymer and combinations thereof.

4. The reagent of claim 3 in which the polymer is selected from the group consisting of collagen, gelatin, serum albumin, globulin, polyethylene glycol, polyethylene oxide, polypropylene glycol, polycaprolactone, polylactide, polyglycolide, polyhydroxyvalerate, polyhydroxybutyrate, carbohydrates, carbohydrate derivatives, non-polyamino acid synthetic polymers and co-polymers and combinations thereof.

5. The reagent of claim 1 in which the polymeric particle is a capsule comprising a polymer shell enclosing a contrast agent capable of reflecting sound waves.

6. The reagent of claim 5 in which the polymer is selected from the group consisting of a polypeptide, a non-synthetic biodegradable polymer, a synthetic biodegradable polymer and combinations thereof.

7. The reagent of claim 6 in which the polymer is selected from the group consisting of collagen, gelatin, serum albumin, globulin, polyethylene glycol, polyethylene oxide, polypropylene glycol, polycaprolactone, polylactide, polyglycolide, polyhydroxyvalerate, polyhydroxybutyrate, carbohydrates, carbohydrate derivatives, non-polyamino acid synthetic polymers and co-polymers and combinations thereof.

8. The reagent of claim 5 in which the polymer shell comprises an inner layer comprising a biodegradable polymer and an outer layer comprising a cross-linked protein.

9. The reagent of any one of claims 2-8 in which the contrast agent is selected from the group consisting of a gas, a liquid and combinations thereof.

10. The reagent of claim 9 in which the contrast agent is a gas selected from the group consisting of air, nitrogen, oxygen, carbon dioxide, a noble gas, ammonia, a halogenated or partially halogenated hydrocarbon, a liquid having a boiling point of less than 37° C. and combinations thereof.

11. The reagent of claim 9 in which the contrast agent is a liquid selected from the group consisting of perfluorocarbons having more than 6 carbon atoms.

12. (canceled)

13. The reagent of claim 1 in which the annexin V is covalently linked to the particle, optionally by way of a linker.

14. The reagent of claim 1 in which the annexin V is non-covalently linked to the particle through an avidin or streptavidin bridge.

15. The reagent of claim 1 comprising a plurality of particles in which the particles have a mean diameter in the range of about 800 nanometers to 10 microns.

16. The reagent of claim 1 in which the polymeric particle capable of reflecting sound waves includes a therapeutic agent.

17. An ultrasound composition useful for imaging apoptosis, comprising a plurality of microcapsules, each of which comprises:
(i) a shell enclosing a hollow core comprising a contrast agent capable of reflecting sound waves and
(ii) an annexin V, wherein the annexin V is substantially permanently linked to the shell.

18. The composition of claim 17 in which the shell comprises an inner layer comprising a biodegradable synthetic polymer and an outer layer comprising a cross-linked protein.

19. The composition of claim 18 in which the biodegradable polymer is selected from the group consisting of polycaprolactone, polylactide, polyglycolide, polyhydroxyvalerate, polyhydroxybutyrate or copolymers thereof.

20. The composition of claim 18 in which the protein is selected from the group consisting of collagen, gelatin, serum albumin, globulin, and combinations thereof.

21. The composition of claim 18 in which the protein is cross-linked with a bi-functional aldehyde or a carbodiimide.

22. The composition of claim 21 in which said bi-functional aldehyde comprises glutaraldehyde.

23. The composition of claim 18 in which the annexin V is covalently linked to the outer layer of the shell, optionally by way of a linker.

24. The composition of claim 18 in which the contrast agent is a liquid or a gas or a mixture thereof.

25. The composition of claim 18 in which the microcapsules further comprise a therapeutic agent.

26. The composition of claim 25 in which the hollow core comprises the therapeutic agent.

27. The composition of claim 25 in which the shell comprises the therapeutic agent.

28. An ultrasound contrast composition useful for imaging apoptosis, comprising a plurality of reagents of claim 1 and a pharmaceutically acceptable excipient, carrier and/or diluent.

29. A composition useful for administering therapeutic agents to cells and/or tissues undergoing apoptosis, comprising a plurality of reagents according to claim 16 and a pharmaceutically acceptable excipient, carrier, and/or diluent.

30. A method of diagnosing and/or imaging a condition characterized by apoptosis, comprising administering to a subject a composition of claim 28 and obtaining an ultrasound image of at least a region of the subject.

31. The method of claim 30 in which the composition is administered via injection and the reagents are permitted to clear from the circulatory system prior to obtaining the image.

32. A method of locally administering a therapeutic agent to an apoptotic cell, comprising administering to a subject a composition according to claim 29 and rupturing the reagents with ultrasonic energy.

33. The method of claim 32 further including ultrasound imaging of said reagents.
